(19) United States
(12) Patent Application Publication
(10) Pub. No.: US 2018/0271058 A1
(43) Pub. Date: Sep. 27, 2018

(54) TRACKING AND LOCATION SYSTEM FOR PETS

(71) Applicant: Reynaldo Valdez, Donna, TX (US)

(72) Inventor: Reynaldo Valdez, Donna, TX (US)

(21) Appl. No.: 15/974,646

(22) Filed: May 8, 2018

Related U.S. Application Data
(60) Provisional application No. 62/468,580, filed on Mar. 8, 2017.

(51) Int. Cl. A01K 11/00; A61B 5/00; A61B 5/11; G01S 5/00

(52) U.S. Cl. CPC A01K 11/008; A61B 5/0031; G06K 2017/0045; G01S 5/0027; A61B 5/1112

(57) ABSTRACT

A tracking and location system having an applicator including a syringe with a needle, a tracking unit having a chip, a wireless transponder, and a power supply configured to power the tracking unit. The syringe includes the needle configured to be inserted into and through skin and eject the tracking unit therethrough. The tracking unit includes the chip and the wireless transponder which are in communication with a software application hosted on a mobile device. A location of an animal with the tracking unit implanted, preferably a pet, is viewable on a display of the mobile device.

### CROSS REFERENCE TO RELATED APPLICATION

The present application is related to and claims priority to U.S. Provisional Patent Application No. 62/468,580 filed Mar. 8, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of tracking means and more specifically relates to pet tracking.

### 2. Description of Related Art

One in three pets will get lost within their lifetime. According to one of the first national studies on lost pets, fifteen percent of pet owners reported a lost cat or dog in the last five years. With GPS, the ability to find lost pets increases the chances the pet will be returned to their family. Live GPS devices offer the greatest chance of reuniting pets with families. It is unfortunately common that cats, dogs, and other pets are lost or stolen. A lost pet may be picked up by a pound or shelter, and it can cost an exorbitant amount of money to retrieve the pet. Pets may be stolen by other individuals and a pet owner may have no idea where his/her cat or dog has gone. It is nearly impossible to track an outdoor pet's location at all times. A suitable solution is desired.

U.S. Pub. No. 2013/0285815 to Johnny Jones II relates to an animal tracking system. The described animal tracking system is for remotely monitoring animal positioning. The system includes an implant device, a global positioning system, a network, one or more remote servers, and at least one wireless computing device. The implant device acts as a GPS receiver, calculating the animal's position and then transmitting the results to the remote server where they are stored in a database. Owners can remotely access the database via a software application installed on a wireless computing device. Users can select an acceptable roaming range for the animal, an acceptable range between the animal and other animals being tracked, a lack of movement, or substantial changes in body temperature. Additionally, the implant device can directly connect with the wireless computing device via a Bluetooth connection. In this way, the computing device can access or transfer data regarding owner contract info, and an animal's medical history.

### BRIEF SUMMARY OF THE INVENTION

In view of the foregoing disadvantages inherent in the known tracking means art, the present disclosure provides a novel tracking and location system for pets. The general purpose of the present disclosure, which will be described subsequently in greater detail, is to provide a tool and method of inserting a tracking chip under a pet's skin for tracking purposes.

A tracking and location system is disclosed herein. The tracking and location system includes an applicator including a syringe with a needle, a tracking unit having a chip, a wireless transponder, and a power supply configured to power the tracking unit. The syringe includes the needle configured to be inserted into and through skin and eject the tracking unit therethrough. The tracking unit includes the chip and the wireless transponder. The tracking unit is configured to be injected under the skin of an animal by the syringe. The chip and the wireless transponder are in communication with a software application hosted on a mobile device. A location of the animal, preferably a pet, is viewable on a display of the mobile device.

According to another embodiment, a method of tracking a pet is also disclosed herein. The method of tracking a pet includes providing a tracking and location system comprising an applicator including a syringe with a needle, a tracking unit having a chip, a wireless transponder, and a power supply configured to power the tracking unit; removing a sterile cover; inserting the needle into skin of an animal; injecting the tracking unit; communicating with the tracking unit; tracking the pet; and removing the tracking unit.

A kit is also disclosed herein including a set of instructions, an applicator, at least one tracking unit, and a sterile carrying case.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a tracking and location system for pets, constructed and operative according to the teachings of the present disclosure.

FIG. 1 is a perspective view of the tracking and location system during an ‘in-use’ condition, according to an embodiment of the disclosure.

FIG. 2 is a perspective view of the tracking and location system of FIG. 1, according to an embodiment of the present disclosure.

FIG. 3 is a perspective view of the tracking and location system of FIG. 1, according to an embodiment of the present disclosure.

FIG. 4 is a perspective view of the tracking and location system of FIG. 1, according to an embodiment of the present disclosure.

FIG. 5 is a flow diagram illustrating a method of use for the tracking and location system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a tracking means and more particularly to a tracking and location system for pets as used to improve pet tracking using a tracking unit which may be inserted under skin of a pet using a kit.

Generally, the tracking and location system provides pet owners with a way to constantly monitor their cat, dog, or other animal's location. It utilizes a syringe to inject the chip under the skin, preferably on the back of the neck to avoid discomfort for the animal. It features a pre-packaged sterile cork placed over the 20-gauge needle to prevent any cross-contamination from occurring before the chip is inserted. It communicates with a smartphone, tablet, and the like, via a software application which allows the pet owner to track and identify a pet's location at all times.

The present invention eliminates the worry of a pet being permanently lost and/or stolen. Tracking and location system is a tracking and location system for pets. The tracking and location system is comprised of a sterile syringe capable of inserting a tracking chip/unit underneath the skin. The tracking chip is comprised of a wireless transponder capable of communicating via a software program installed on a smartphone, tablet, and the like. The syringe features a 20-gauge needle and sanitary packaging for easy, convenient insertion. The syringe can feature a sterile cork to prevent any contact before the tracking unit is placed underneath a pet's skin. Users can remove the cork, insert the needle into the pet, and then inject the tracking unit. The process is performed on the back of a pet's neck in order to eliminate discomfort. Pet owners can then track their pet via the unit and the software application. The system is intended to prevent pets from being lost or stolen.

Upon reading this specification, it should be appreciated that, under appropriate circumstances, considering such issues as user preferences, design preference, structural requirements, marketing preferences, cost, available materials, technological advances, etc., other tracking arrangements such as, for example, transmitters, receivers and other chips and wireless communication means, etc., may be sufficient.

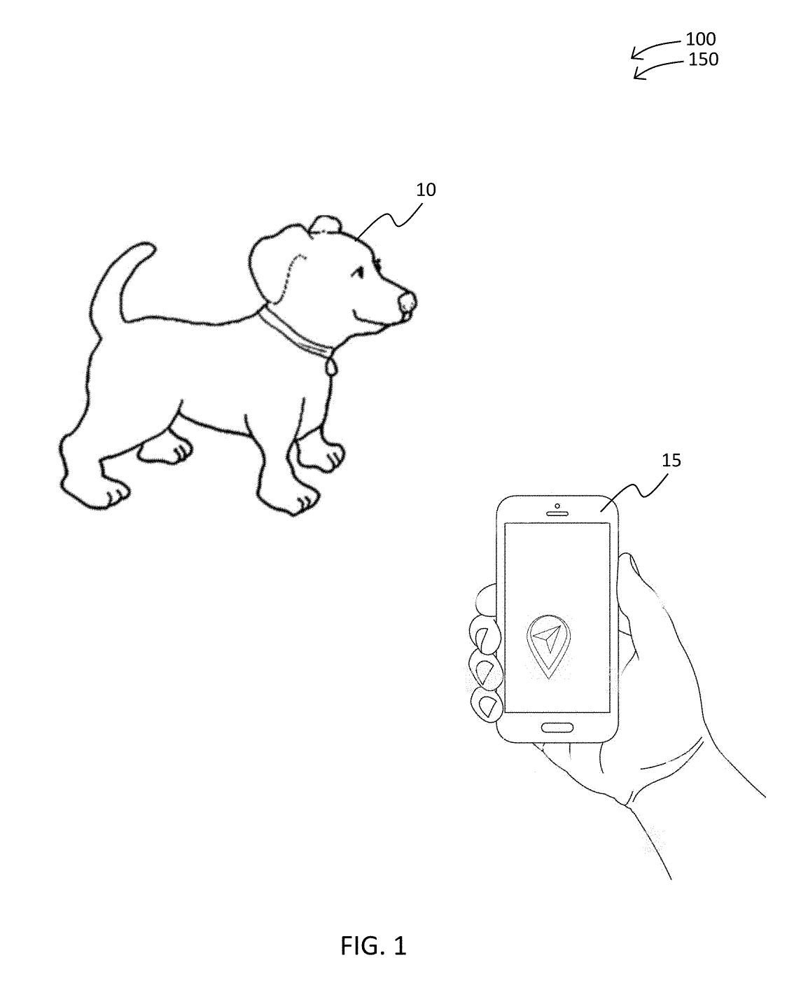

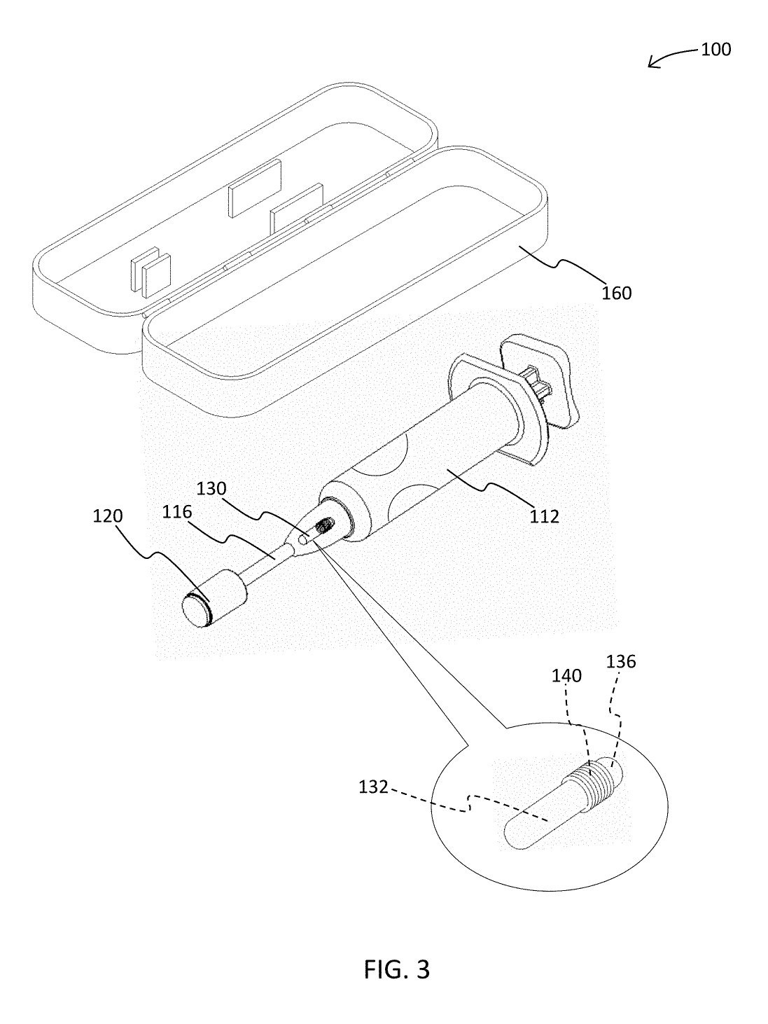

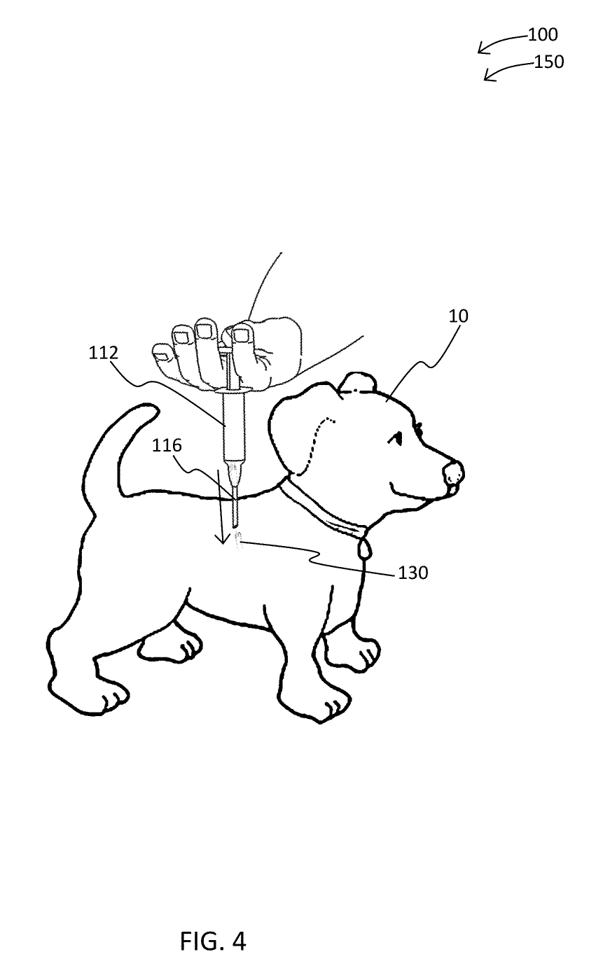

Referring now more specifically to the drawings by numerals of reference, there is shown in FIGS. 1-5, various views of a tracking and location system 100. FIG. 1 shows a tracking and location system 100 during an ‘in-use’ condition 150, according to an embodiment of the present disclosure. As illustrated, the tracking and location system 100 may include an applicator 110 including a syringe 112 with a needle 116, a tracking unit 130 having a chip 132, a wireless transponder 136, and a power supply 140 configured to power the tracking unit 130. The syringe 112 includes the needle 116 which is configured to be inserted into and through skin and eject the tracking unit 130 therethrough. The tracking unit 130 includes the chip 132 and the wireless transponder 136. The tracking unit 130 is configured to be injected under the skin of an animal 10 by the syringe 112. The chip 132 and the wireless transponder 136 are in communication with a software application hosted on a mobile device 15. A location of the animal 10, preferably a pet, is viewable on a display of the mobile device 15.

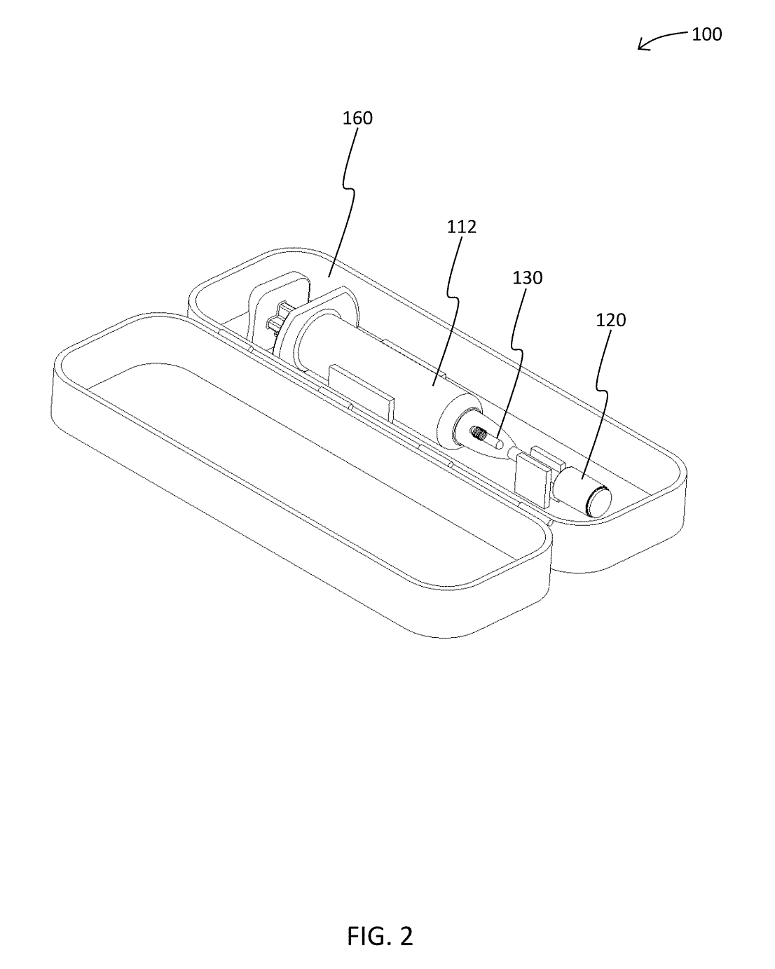

FIGS. 2-4 show views of the tracking and location system 100 of FIG. 1, according to an embodiment of the present disclosure. As above, the tracking and location system 100 may include the applicator 110 and the tracking unit 130 to be used for tracking animals 10, in particular pets. The applicator 110 includes a syringe 112 having a needle 116. In a preferred embodiment, the syringe 112 comprises a 20-gauge needle. The applicator 110 is configured to be used by a pet-owner. The applicator 110 further includes a sterile cover 120. The sterile cover 120 may be cork and is configured to be placed over the 20-gauge needle 116 to prevent cross-contamination from occurring. The applicator 110 is sterile and allows a user to apply the tracking unit 130 in a safe and sanitary manner.

The tracking unit 130 operates as a GPS receiver and is configured to transmit location data to the mobile device 15. The location data includes GPS coordinates viewable on the mobile device 15. The GPS coordinates are updated in real-time to provide updated the location data as desired. The tracking unit 130 is configured to receive a signal from a remote the mobile device 15 and provide the location data as needed. The tracking unit 130 further includes a memory for temporarily storing location data and a processor. The wireless transponder 136 transmits a pre-determined frequency and receives a specific frequency allowing for communication. The tracking unit 130 is configured to be injected under the skin on a back-portion of a neck of the pet 10. However, the tracking unit 130 may be injected at any location using the applicator 110. Those with ordinary skill in the art will now appreciate that upon reading this specification and by their understanding the art of GPS and tracking means as described herein, methods of tracking a pet will be understood by those knowledgeable in such art.

According to one embodiment, the tracking and location system 100 may be arranged as a kit 105. In particular, the tracking and location system 100 may further include a set of instructions, an applicator 110, at least one tracking unit 130, and a sterile carrying case 160. The instructions 107 may detail functional relationships in relation to the structure of the tracking and location system 100 such that the tracking and location system 100 can be used, maintained, or the like, in a preferred manner.

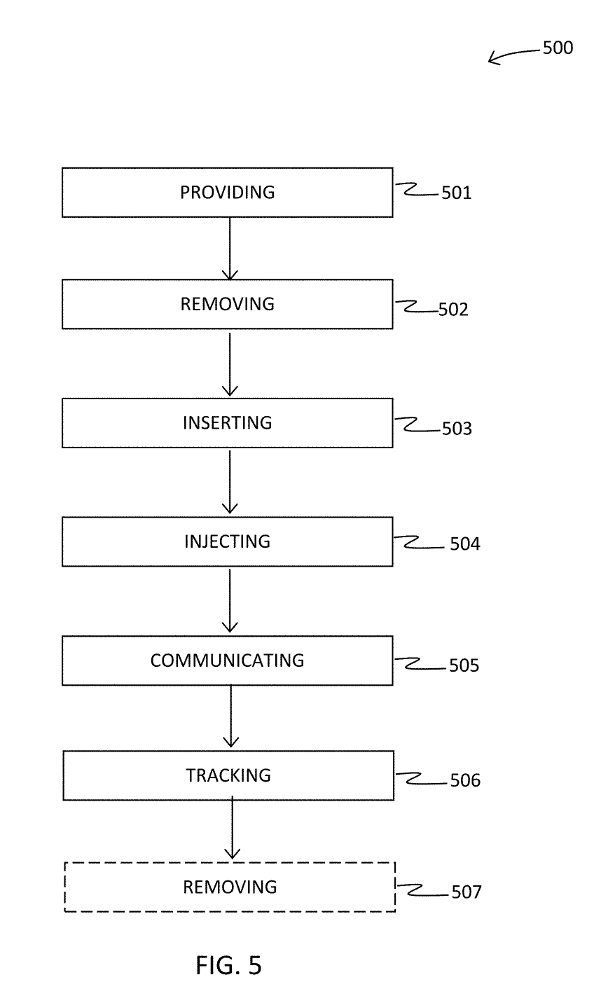

FIG. 5 is a flow diagram illustrating a method for method of tracking a pet 500, according to an embodiment of the present disclosure. In particular, the method of tracking a pet 500 may include one or more components or features of the tracking and location system 100 as described above. As illustrated, the method of tracking a pet 500 may include the steps of: step one 501, providing a tracking and location system 100 comprising an applicator 110 including a syringe 112 with a needle 116, a tracking unit 130 having a chip 132, a wireless transponder 136, and a power supply 140; step two 502, removing a sterile cover 120; step three 503, inserting the needle 116 into skin of an animal 10; step four 504, injecting the tracking unit 130; step five 505, communicating with the tracking unit 130; step six 506, tracking the pet 10; step seven 507, removing the tracking unit 130.

It should be noted that step seven 507 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for method of tracking a pet, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A tracking and location system comprising:
an applicator including;
a syringe with
a needle;
a tracking unit having;
a chip;
a wireless transponder; and
a power supply configured to power said tracking unit;
wherein said syringe includes said needle configured to be inserted into and through skin and eject said tracking unit therethrough;
wherein said tracking unit includes said chip and said wireless transponder;
wherein said tracking unit is configured to be injected under said skin of an animal by said syringe;
wherein said chip and said wireless transponder are in communication with a software application hosted on a mobile device; and
wherein a location of said animal is viewable on a display of said mobile device.

2. The tracking and location system of claim 1, wherein said syringe comprises a 20-gauge said needle.

3. The tracking and location system of claim 1, wherein said animal is a pet.

4. The tracking and location system of claim 1, wherein said tracking unit operates as a GPS receiver and is configured to transmit location data to said mobile device.

5. The tracking and location system of claim 4, wherein said location data includes GPS coordinates.

6. The tracking and location system of claim 5, wherein said GPS coordinates are updated in real-time to provide updated said location data as desired.

7. The tracking and location system of claim 6, wherein said tracking unit is configured to receive a signal from a remote said mobile device.

8. The tracking and location system of claim 1, wherein said tracking unit further includes a memory for storing location data.

9. The tracking and location system of claim 8, wherein said tracking unit further includes a processor.

10. The tracking and location system of claim 1, wherein said applicator is configured to be used by a pet-owner.

11. The tracking and location system of claim 1, wherein said applicator further includes a sterile cover.

12. The tracking and location system of claim 11, wherein said sterile cover comprises cork to be placed over said 20-gauge needle to prevent cross-contamination from occurring.

13. The tracking and location system of claim 1, wherein said wireless transponder transmits a pre-determined frequency and receives a specific frequency.

14. The tracking and location system of claim 1, wherein said applicator is sterile.

15. The tracking and location system of claim 3, wherein said tracking unit is configured to be injected under said skin on a back-portion of a neck of said pet.

16. The tracking and location system of claim 1, further includes a sterile carrying case.

17. A tracking and location system, the tracking and location system comprising:
an applicator including;
a syringe with
a needle;
a tracking unit having;
a chip;
a wireless transponder; and
a power supply configured to power said tracking unit;
wherein said syringe includes said needle configured to be inserted into and through skin and eject said tracking unit therethrough;
wherein said syringe comprises a 20-gauge said needle;
wherein said tracking unit includes said chip and said wireless transponder;
wherein said tracking unit is configured to be injected under said skin of an animal by said syringe;
wherein said animal is a pet;
wherein said tracking unit operates as a GPS receiver and is configured to transmit location data to said mobile device;
wherein said chip and said wireless transponder are in communication with a software application hosted on a mobile device;
wherein a location of said animal is viewable on a display of said mobile device;
wherein said location data includes GPS coordinates;
wherein said GPS coordinates are updated in real-time to provide updated said location data as desired;
wherein said tracking unit is configured to receive a signal from a remote said mobile device;
wherein said tracking unit further includes a memory for storing location data;
wherein said tracking unit further includes a processor;
wherein said applicator is configured to be used by a pet-owner;
wherein said applicator further includes a sterile cover;
wherein said sterile cover comprises cork to be placed over said 20-gauge needle to prevent cross-contamination from occurring;
wherein said wireless transponder transmits a pre-determined frequency and receives a specific frequency;
wherein said applicator is sterile; and
wherein said tracking unit is configured to be injected under said skin on a back-portion of a neck of said pet.

18. The tracking and location system of claim 17, further comprising set of instructions; and
an applicator;
at least one tracking unit;
a sterile carrying case; and
wherein the tracking and location system is arranged as a kit.

19. A method of tracking a pet, the method comprising the steps of:
providing a tracking and location system comprising:
an applicator including;
a syringe with
a needle;
a tracking unit having;
a chip;
a wireless transponder; and
a power supply configured to power said tracking unit;
removing a sterile cover;
inserting said needle into skin of an animal;
injecting said tracking unit;
communicating with said tracking unit; and
tracking said pet.

20. The method of claim 19, further comprising the steps of:
removing said tracking unit.
